(19) United States
(12) Patent Application Publication
(10) Pub. No.: US 2007/0098732 A1
(43) Pub. Date: May 3, 2007

(54) Immunologic compounds for prevention, protection, prophylaxis or treatment of immunological disorders, infections and cancer

(75) Inventors: Denis Claude Roy, Laval (CA); Marc Vaillancourt, Chateauguay (CA); Luc Villeneuve, Montreal (CA)

Correspondence Address: CROWELL & MORING LLP;INTELLECTUAL PROPERTY GROUP, P.O. BOX 14300, WASHINGTON, DC, 20044-4300, US

(73) Assignees: Universite De Montreal; Hopital Maisonneuve-Rosemont

(21) Appl. No.: 10/581,718

(22) PCT Filed: Dec. 2, 2004
(86) PCT No.: PCT/CA04/02070

§ 371 Date: Jun. 22, 2006

Related U.S. Application Data
(60) Provisional application No. 60/526,977, filed on Dec. 5, 2003.

(51) Int. Cl. A61K 39/00; A61K 31/365; A61K 31/353

(52) U.S. Cl. 424/184.1; 514/453; 514/454

(57) ABSTRACT

The present invention relates to the use of PDT-treated cells (whole or fragments thereof) and/or supernatant thereof in the preparation of vaccines for immunoprotection or immunomodulation. More particularly, the use of PDT-treated cells (whole or fragments thereof) and/or supernatant thereof in the preparation of an immunologic compound for prevention, protection, prophylaxis or treatment of an immunological disorder, infection and/or a cancer in an individual, which comprises treatment of the individual cells or components thereof with a photoactivatable molecule selected from the group consisting of compounds I to XVIII, wherein the photoactivatable molecule is activated by a light of appropriate wavelength, thereby activating the photoactivatable molecule and causing prevention, protection, prophylaxis or treatment of the immunological disorder, infection and/or a cancer.

### BACKGROUND OF THE INVENTION

(a) Field of the Invention

The present invention relates to the use of PDT-treated cells (whole or fragments thereof) and/or supernatant thereof in the preparation of vaccines for immunoprotection or immunomodulation. The PDT-treated cells and lysate thereof are prepared by treating cells with photoactivatable molecules, such as 2′-(6-amino-4,5-dibromo-3-imino-3H-xanthen-9-yl)-benzoic acid methyl ester hydrobromide (hereinafter referred to as TH9402) and derivatives thereof (as described in International Patent Application published under No. WO 02/079183) and activating these molecules with light. The main characteristic of these molecules is their ability to accumulate into and eradicate cells once activated, such cells include, without limitation, immune cells, cancer cells, infected cells, without affecting progenitor stem cells. This particularity is of great interest since repetitive extracorporeal treatment of blood cells and their reinjection into the bloodstream has a limited effect on lymphocytes, as mainly activated cells will be eradicated, and resting cells are spared in higher proportions. While intercalating agent such as 8-methoxypsoralen necessitate the usage of UV irradiation, photoactivatable molecules of the present invention (TH9402 and derivatives thereof) use visible light for their activation, thereby reducing the risk of mutagenic effects. Other agents such as Intercept™ are also intercalating agents. Since the photoactivatable molecules of the present invention (TH9402 and derivatives thereof) do not accumulate in cell nuclei, they have a low potential of causing DNA damage, mutation and/or carcinogenesis.

(b) Brief Description of the Prior Art

Photodynamic Therapy (PDT) uses chemical compounds activated by various light/irradiation devices. Cytotoxicity of the activated species leads to the eradication of cells and to the presentation of their antigens to stimulate an immune response or cause immunomodulation. Photodynamic therapy could also affect targeted cells by inducing apoptosis. Apoptotic cells are known for their capacity to present their own antigens to professional antigen presenting cells, such as dendritic cells. Such antigen presentation can lead to the development of a response of the immune system toward these immunizing antigens. Many reports have demonstrated the usefulness of adjuvants to boost the immune response toward the killed cells. Among others, pertinent references such as works by Korbelik (Korbelik et al, Laser Med. Surg, 14 (1996), 329-334, Can. Res., 56, (1996) 5647-5565; Chen et al, SPIE, 394 (2000), 26-32), as well as Nordquist et al (International Patent Applications published under Nos. WO 96/31237 and WO 99/47162A1) have demonstrated the usefulness of such an approach. Moreover, the usage of oxygenated species in blood components has been described previously using ozone as the chemical agent in conjunction with irradiation (Zee et al, U.S. Pat. No. 4,632,980; Fish et al, U.S. Pat. No. 4,831,268, Mueller et al, U.S. Pat. No. 4,968,483). Photodynamic Therapy has also been extensively described in “Photosensitizing Compounds: their Chemistry, Biology and Clinical uses” (1989, John Wiley & Sons, Chichester, UK, ISBN 0471923087). Many other pertaining references relating to the usage of Photosensitizers in the treatment of tumor masses combined with antibodies (Levy et al, U.S. Pat. Nos. 5,095,030 & 5,283,225) as well as ligands and antibodies (Pendry et al, U.S. Pat. No. 5,241,036). Autoimmune vaccines have been described by Bolton, A.E. (U.S. Pat. No. 6,204,058B1) (International Patent Application published under No. WO 98/07436) on which Rheumatoid Artlritis is treated using leulkocytes with increased expression of specific antigens by oxidizing agents, UV irradiation and high temperature.

Extracorporeal Photopheresis has been described as a successful therapy for the treatment of Hepatitis C, in combination with other means such as Interferon alpha (O'Brien, C. B. International Patent Application published under No. WO 97/376542; McLaughlin S. N. et al, International Patent Application published under No. WO 97/36634), as well as in the treatment of other illnesses mediated by undesired activated immune cells (McLaughlin et al, U.S., Pat. No. 5,984,887 and Bisaccia et al, U.S. Pat. No. 5,426,116). Other studies have been reported regarding the usage of extracorporeal Photopheresis in indications such as organ rejection (Lehrer et al, 2001, The journal of Heart and Lung transplantation, November, 1133-1136; Rosa et al, 1992, Transplantation, 4(53), 808-815; Barr et al, 1998, The New England Journal of Medicine, 339(4), 1744-1751, Barr et al, 2000, Clinical Transplantation, 14, 162-166) as well as for the efficacious treatment of Graft-versus-Host-Disease (Perotti et al, 1999, Haematologica, 84, 237-241; Amico et al, 1997, British Journal of Hematology, 97, 848-854; Rossetti et al, 1995, Transplantation, 59(1), 149-151; Gorgun et al, 2002, Immunobiology, 100(3), 941-947). The indications for the usage of extracorporeal Photophereses is reviewed by Ratanatharathom et al. in Bone Marrow Transplantation (2001, 28, 121-129).

U.S. Pat. No. 5,651,993 (Edelson et al.) teaches other methods for modifying the immune response of a mammal to a specific antigen using irtadication of a leukocyte preparation followed by a adding a mixture of autologous peptides. U.S. Pat. No. 5,147,289 (Edelson) teaches a method of non-specifically enhancing the immune response of a mammal to an antigen which includes withdrawing leukocyte from the mammal, altering the leukocyte cells, and returning the leukocyte cells to the mammal. None of these patents teaches the preparation of a compound for the immunological protection of mammal against infections and cancers.

It would be highly desirable to be provided with the use of PDT-treated cells and supeniatant thereof in the preparation of immunologic compounds for prevention, protection, prophylaxis or treatment of immunological disorders, infections and/or a cancers in an individual.

### SUMMARY OF THE INVENTION

In accordance with the present invention, there is provided the use of PDT-treated cells (whole or fragments thereof) and/or supernatant thereof in the preparation of an immunologic compound for prevention, protection, prophylaxis or treatment of an immunological disorder, infection and/or a cancer in an individual, which comprises treatment of said individual cells or components thereof with a photoactivatable molecule selected from the group consisting of: 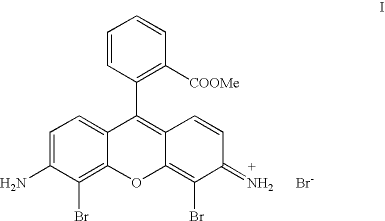
4,5-dibromorhodamine 123 hydrobromide (2′-(6-amino-4,5-dibromo-3-imino-3H-xanthen-9-yl)-benzoic acid methyl ester hydrobromide), also called TH9402, 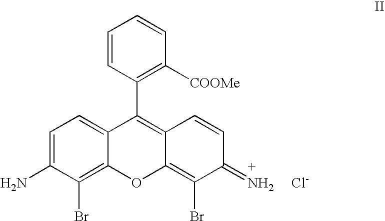
4,5-dibromorhodamine 123 hydrochloride (2′-(6-amino-4,5-dibromo-3-imino-3H-xanthen-9-yl)-benzoic acid methyl ester hydrochloride), 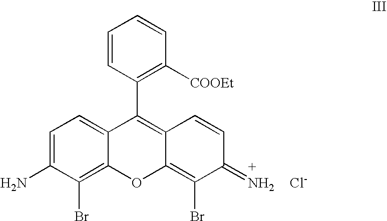
4,5-dibromorhodamine 110 ethyl ester hydrochloride (2′-(6-amino-4,5-dibromo-3-imino-3H-xanthen-9-yl)benzoic acid ethyl ester hydrochloride), 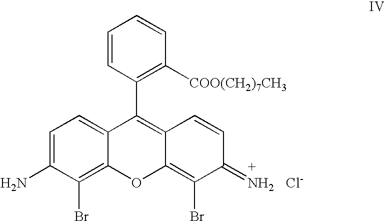
4,5-dibromorhodamine 110 octyl ester hydrochloride (2′-(6-amino-4,5-dibromo-3-imino-3H-xanthen-9-yl)benzoic acid octyl ester hydrochloride), 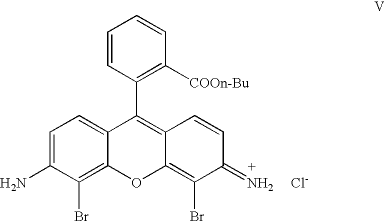
4,5-dibromorhodamine 110 n-butyl ester hydrochloride (2′-(6-amino-4,5-dibromo-3-imino-3H-xanthen-9-yl)benzoic acid n-butyl ester hydrochloride), 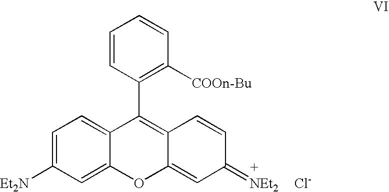
rhodamhie B n-butyl ester hydrochloride (2′-(6-dietiyl amino-3-diethyl imino-3H-xanthen-9-yl)benzoic acid n-butyl ester hydrochloride), 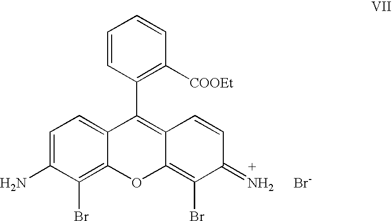
4,5-dibromorhodamine 110 ethyl ester hydrobromide (2′-(6-amino-4,5-dibromo-3-imino-3H-xanthen-9-yl)benzoic acid ethyl ester hydrobromide), 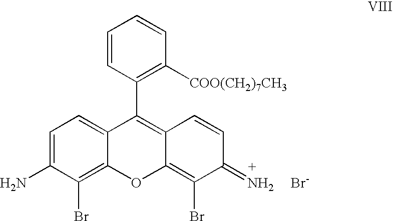
4,5-dibromorhodamine 110 octyl ester hydrobromide (2′-(6-arnino-4,5-dibromo-3-imnino-3H-xanthen-9-yl)benzoic acid octyl ester hydrobromide), 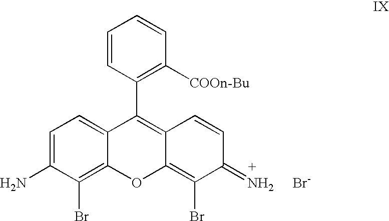
4,5-dibromorhodamine 110 n-butyl ester hydrobromide (2′-(6-amino-4,5-dibromo-3-imino-3H-xanthen-9-yl)benzoic acid n-butyl ester hydrobromide), 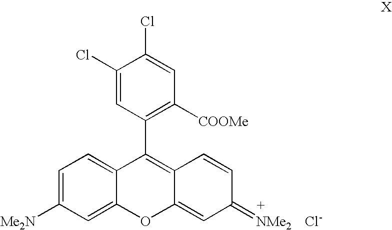
4′,5′-dichlorotetramethylrhodamine (2′-(6-dimethylamino-3-dimethylimino-3H-xanthen-9-yl)-4′,5′-dichloro benzoic acid methyl ester hydrochloride), 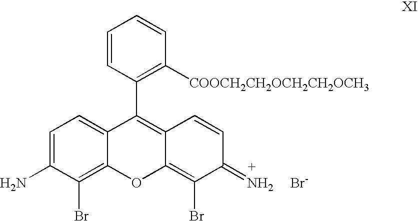
4,5-dibromorhodamine 110 2-(2-methoxy ethoxy)ethyl ester hydrobromide (2′-(6-amino-4,5-dibromo-3-imino-3H-xanthen-9-yl)-benzoic acid 2-(2-methoxy ethoxy) ethyl ester hydrobromide), 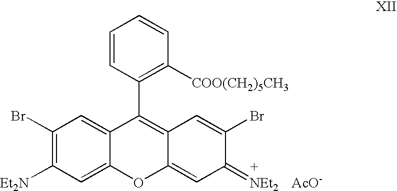
2,7-dibromorhodamine B hexyl ester acetate (2′-(2,7-dibromo-6-diethyl amino-3-diethyl imino-3H-xanthen 9-yl)benzoic acid hexyl ester acetate), 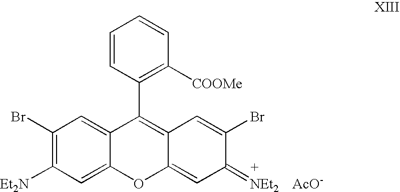
2,7-dibromorhodamine B methyl ester acetate (2′-(2,7-dibromo-6-diethyl amino-3-diethyl imino-3H-xanthen 9-yl)benzoic acid methyl ester acetate), 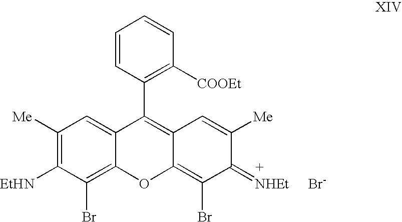
4,5-dibromorhodamine 6G hydrobromide (2′-(4,5-dibromo-2,7-dimethyl-6-ethylamino-3-ethylimino-3H-xanthen-9-yl)benzoic acid ethyl ester hydrobromide), 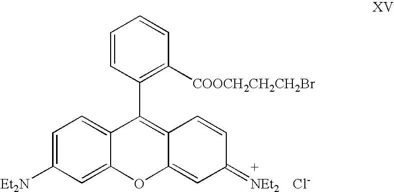
rhodamine B 3-bromopropylester hydrochloride (2′-(6-diethyl amino-3-diethyl imino-3H-xanthen-9-yl)benzoic acid 3-bromopropyl ester hydrochloride), 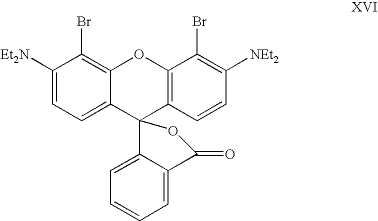
4,5-dibromorhodamine B base (3,3-(4′,5′-dibromo-3′-diethyl amino-6′-diethyl aminoxanthen-9′-yl)-3H-isobenzofuran-1-one), 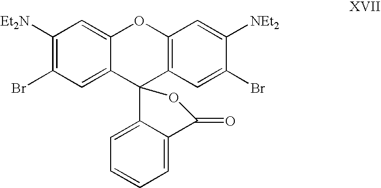
2,7-dibromorhodamine B base (3,3-(2′,7′-dibromo-3′-diethyl amino-6′-diethyl aminoxanthen-9′-yl)-3H-isobenzofuran-1-one) and 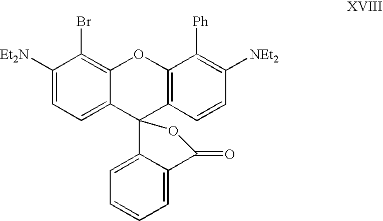
4-bromo-7-phenyl-rhodamine B base (3,3-(4′-bromo-3′-diethyl amino-6′-diethyl amino-5′-phenyl xanthen-9′-yl)-3H-isobenzofuran-1-one)

and wherein said photoactivatable molecule is activated by a light of appropriate wavelength, thereby activating said photoactivatable molecule and causing prevention, protection, prophylaxis or treatment of said immunological disorder, infection and/or a cancer.

Also in accordance with the present invention, there is provided an immunologic vaccine comprising PDT-treated cells (whole or fragments thereof) and/or supernatant thereof, wherein said cells are treated with a photoactivatable molecule of formula (I) as previously defined, in association with a pharmaceutically acceptable carrier. The vaccine of the invention can be used for prevention, protection, prophylaxis or treatment of said immunological disorder, infection and/or a cancer.

Still in accordance with the present invention, there is provided a method of preparing an immunologic compound for prevention, protection, prophylaxis or treatment of an immunological disorder, infection and/or a cancer in an individual, which comprises the steps of:
a) treatment of said individual cells with a photoactivatable molecule of formula (I) as previously defined; and
b) subjecting said cells to a light of appropriate wavelength to activate said photoactivatable molecule, thereby obtaining PDT-treated individual cells (whole or fragments thereof) and/or supernatant thereof.
For the purpose of the present invention the following terms are defined below.
   “PDT-treated cells” means cells which have been treated with a photoactivatable molecule activated by a light of appropriate wavelength;

### BRIEF DESCRIPTION OF THE DRAWINGS

FIG. 1A illustrates a Kaplan-Meier survival analysis of mice after administration of cells treated or not with PDT. There is improved survival for animals receiving PDT treated cells (4×10⁶ T cells)(+PDT) over those receiving non-PDT treated cells (−PDT)(p=0.02).

FIG. 1B illustrates the survival analysis of mice with acute GvHD receiving 0.4×10⁶ treated (+PDT) and untreated (−PDT) T cells obtained from mice also suffering from acute GvHD. Survival of animals receiving 0.4×10⁶ PDT treated T cells (+PDT) is improved (delete:survival) over Control (p=0.04). Survival of animals receiving 0.4×10⁶ PDT treated T cells (+PDT) is also improved over that of animals receiving 0.4×10⁶ untreated T cells (−PDT) (p=0.01).

FIG. 2 illustrates a tumor growth comparison between mice immunized with a supernatant from PDT-treated cells and mice which have not been immunized with such a supernatant. Vaccination with supernatant from PDT-treated cells delayed tumor growth.

FIG. 3 illustrates that tumor-free survival is promoted by immunizing animals with dendritic cells that were coincubated with whole PDT-treated (P815) tumor cells versus mice vaccinated with dendritic cells alone (p<0.01).

### DETAILED DESCRIPTION OF THE INVENTION

Immunological Disorders, Infections and Cancers

In the present invention, there is provided several solutions for prevention, protection and/or prophylaxis or treatment of an immunological disorder, infection and/or a cancer in an individual. In particular, the immunological disorder can be an alloimmune disorder or an autoimmune disorder. The alloimmune disorder can be Graft-versus-Host Disease or an organ rejection. Examples of autoimmune diseases include but are not limited to Rheumatoid Arthritis, Multiple Sclerosis, Scleroderma, Lupus, Autoimmune Hemolytic Anemia, Diabetes Mellitus, Progressive Systemic Sclerosis, Idiopathic Thrombocytopenic Purpura, Psoriasis, Ulcerative Colitis and Crohn's Disease. The infection can be caused by a bacteria, a virus, a parasite, a fungus, a prion, a protozoan or other infection agents. Also, the infection can cause Chagas' Disease. Examples of viruses include but are not limited to Human Immunodeficiency Virus (HIV), Hepatitis B Virus (HBV), Hepatitis C Virus (HCV), Human Herpes Virus Type I or II, and Varicella Zoster. Example of cancers include but are not limited to solid tumors and hematologic tumors. The solid tumors can be of breast cancer, lung cancer, gastrointestinal cancer, skin cancer or of genitourinary, neurological, head and neck or musculoskeletal origin. The hematologic tumors can be lymphomas, leukemias, myelomas, myelodysplasias or plasma cell dyscrasias.

Immunologic Compounds of the Present Invention

The following specific photoactivatable molecules are particularly preferred: 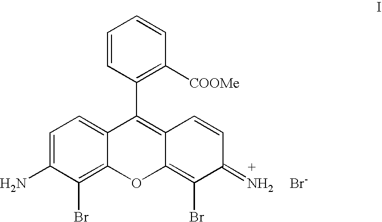
4,5-dibromorhodamine 123 hydrobromide (2′-(6-amino-4,5-dibromo-3-imino-3H-xanthen-9-yl)-benzoic acid methyl ester hydrobromide) also called TH9402, and 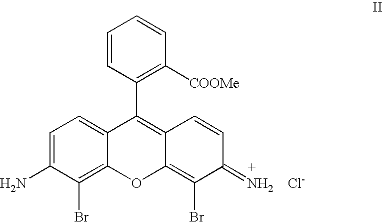
4,5-dibromorhodamine 123 hydrochloride (2′-(6-amino-4,5-dibromo-3-imino-3H-xanthen-9-yl)-benzoic acid methyl ester hydrochloride).

The photoactivatable molecules of the invention are activatable by a light of appropriate wavelenght which is preferably ranging from about 400 to about 800 nm and more preferably from about 450 to about 600 nm.

PDT of the Present Invention

PDT is preferably based on the exposition of the photoactivatable molecules of the invention to visible light, which can produce free radical species. Cationic rhodamines such as TH9402 have been shown to specifically accumulate in mitochondria and the production of free radicals leads to mitochondria collapse. Accumulation is increased in activated cells, making the effect of those rhodamines a particularly attractive therapy for activated cells in autoimmune diseases. Also, exposure of the immune system cells to other immunologic cells reacting toward host tissues or the transplanted,organ, cancer cells, infected cells or other undesirable cells treated by PDT is particularly attractive for vaccination and extracorporeal photopheresis leading to beneficial immunomodulation.

PDT-treated cells and/or lysate thereof, including cell products released from these cells after PDT treatment with the photoactivatable molecules of the present invention, can be used either alone or in association with adjuvant in order to generate specific immune responses from an individual. These vaccines can be used to treat individuals suffering from autoimmune diseases such as, but not limited to: Rheumatoid Arthritis, Multiple Sclerosis, Scleroderma, Lupus erythematosus, Diabetes, Autoimmune Hemolytic Anemia, Diabetes Mellitus, Progressive Systemic Sclerosis, Idiopathic Thrombocytopenic Purpura, Psoriasis, Ulcerative Colitis, Crohn's Disease as well as to illnesses evading the immune system such as, but not limiting to: Acquired Immunodeficiency Syndrome (AIDS), Human Immunodeficiency Virus (HIV), Hepatitis C Virus (HCV), Hepatitis B Virus (HBV), Human Herpes Virus Type I or II, and Varicella Zoster. Moreover, these vaccines can also lead to the improvement of cancer treatments by inducing an immune response to the evading cancer cells. This could lead to the physical destruction of tumor masses induced by a directed immune response.

In the present invention, the treatment of the individual cells is effected ex vivo, in vitro or in vivo. Preferably, the treatment is effected ex vivo by perfusion. Extracorporeal treatment of cells can also be used for the repetitive injection of a portion of PDT treated blood cells, which are then reinjected into the individuals. This treatment is used for the improvement of acute and chronic conditions such as, but not limited to, Graft-versus-Host Disease, organ rejection, debilitating diseases caused by an autoimmune reaction such as, but not limited, to Rheumatoid Arthritis, Multiple Sclerosis, Scleroderma, Lupus, Type I and II Diabetes, Autoimmune Hemolytic Anemia, Diabetes Mellitus, Progressive Systemic Sclerosis, Idiopathic Thrombocytopenic Purpura, Psoriasis, Ulcerative Colitis and Crohn's Disease. This treatment also enhances the immune response against treated cells by the individual.

More specifically, in a preferred vaccine of the present invention, when PDT treated lymphoma cells or lymphoma cells supernatant obtained after exposure to PDT, are injected into mice, a significant decrease of lymphoma formation is observed. Such alleviation has been demonstrated using the T-cell lymphoma cell line EL-4. EL-4 lymphoma cells undergoing PDT with TH9402 and light rapidly proceed to programmed cell death, apoptosis and/or necrosis (see Example 2).

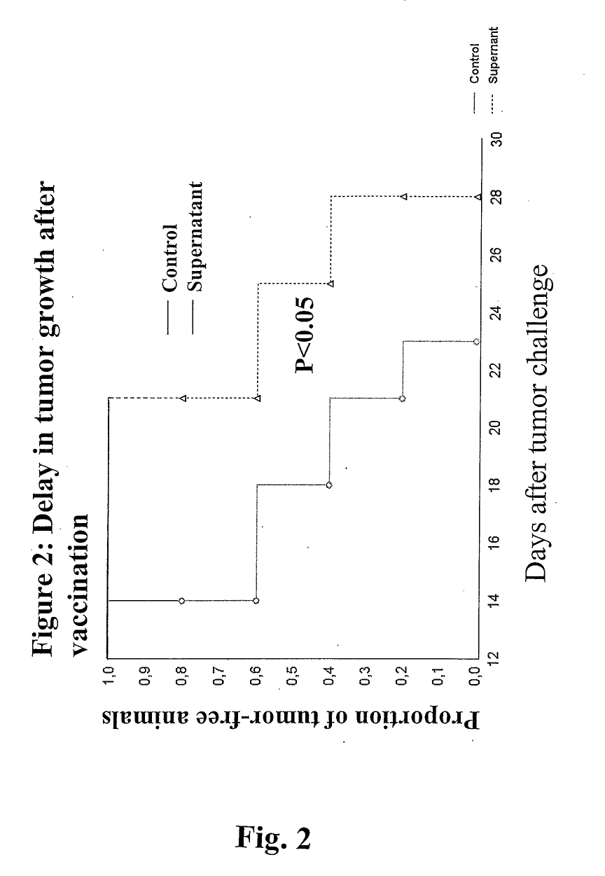

Repetitive subcutaneous injections of the supernatant from PDT treated EL-4 cells, or from irradiated PDT treated EL-4 cells in mice for 4 weeks followed by injection of non-treated cells clearly indicate that mice are demonstrating delayed growth of lymphoma. In contrast, non-vaccinated mice develop earlier lymphoma cell growth leading to death (FIG. 2).

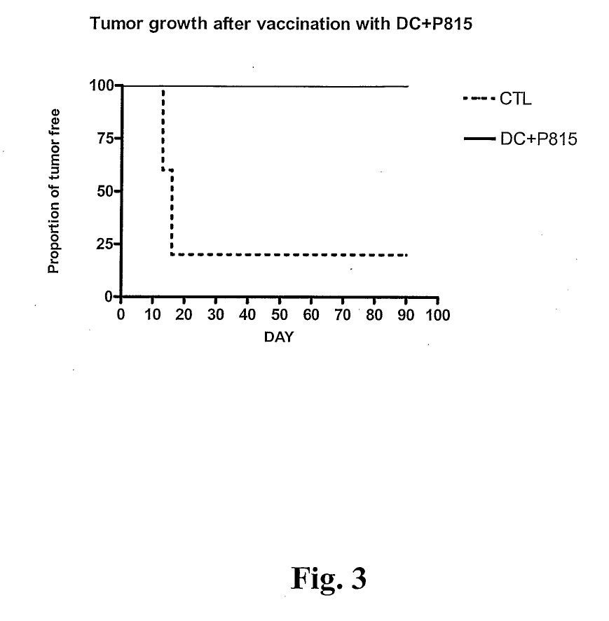

Similarly, P815 tumor cells (mastocytoma) were PDT-treated and coincubated with antigen presenting cells such as dendritic cells and then used to immunize mice in a repetitive fashion (see Example 3). When such animals immunized against PDT-treated P815 tumor cells were injected with fresh P815 tumor cells, these mice demonstrated improved tumor-free survival over mice immunized with unnanipulated dendritic cells (FIG. 3).

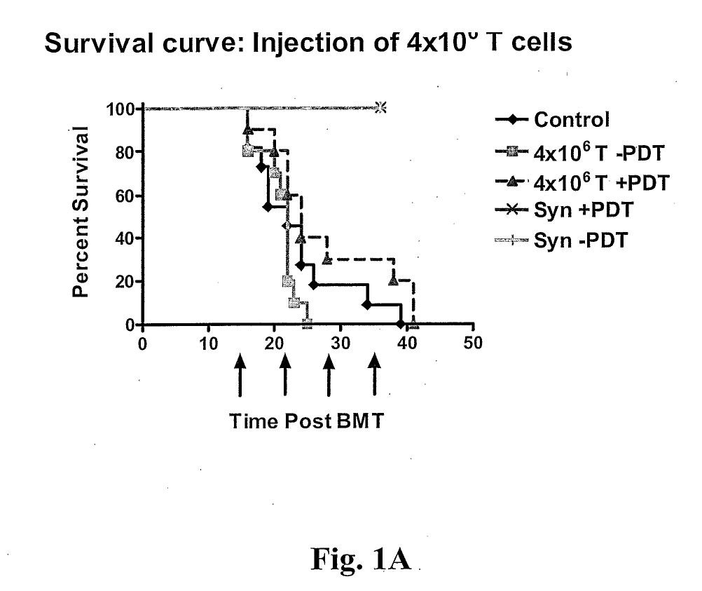

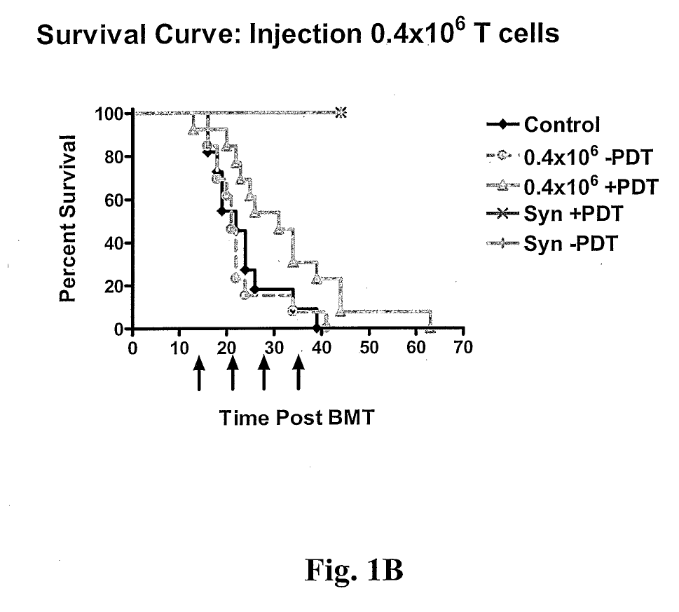

Immunomodulation is believed to be performed through the unique potential of the immune system to develop an aggressive and specific response toward dysfimctional or dying cells. Antigen presenting cells process and present antigens based on their propensity to process antigens from cells undergoing programmed cell death or apoptosis, and also from cells damaged or dying from necrosis. Since mainly activated cells will be eradicated by photoactivatable molecules of the present invention (TH9402 and derivatives thereof), analysis of the cell population undergoing apoptosis and necrosis has been evaluated. Data indicates that B-cells, dendritic cells and activated T-cells (il faudrait enlever NK cells ou mettre “possibly NK cells”) among others, are rapidly eliminated. This advantage is exploited by inducing the immune system to produce an immune response against autoreactive T-cells. This property has been used in mice models and humans developing GvHD. Peripheral blood cells from individuals with GvHD are harvested, usually by leukopheresis, and exposed to PDT. These treated cells are then reinfused into the individual and this procedure is repeated at regular intervals. This treatment leads to improvement of GvHD that occurs after stem cell transplantation. PDT using photoactivatable molecules of the present invention (TH9402 and derivatives thereof) is able to prevent the development or treat GvHD in mice that received PDT-treated cells at regular intervals. This leads to improved survival of mice infused with PDT-treated cells. In contrast, mice receiving either non-PDT treated cells or inedia alone are developing GvHD leading to death. This is also shown in FIGS. 1A and 1B using Kaplan-Meier survival analysis.

The present invention will be more readily understood by referring to the following examples which are given to illustrate the invention rather than to limit its scope.

### EXAMPLE 1

### Treatment of GvHD in Mice

One preferred embodiment of the present invention is to use whole cells exposed to photoactivatable molecules of the present invention (TH9402 and derivatives thereof) with PDT and also cell lysates generated after such treatment.

Materials and Methods

Extracorporeal Phototherapy:

Mice. The following strains of mice were purchased from The Jackson Laboratory: C57BL/6 (B6) (H-2^{b}), B10BR (H-2^{k}). Mice were bred and housed in specific pathogen-free conditions at the Guy-Bemier Research Centre according to the standards set by the Canadian Committee for Animal Protection. All mice were used between 6-10 weeks of age.

Cell Transplantation. Bone marrow cells were harvested from tibias and femurs of donor mice, T cell depleted and transplanted in recipient mice. Briefly, cells were suspended at a concentration of 1×10⁷ cells /ml in RPMI 1640 supplemented with 5% FBS, 100 U/ml penicillin G, and 100μg/ml streptomycin, and incubated with rabbit anti-mouse T cells (Thyl) antiserum (Cedarlane Labs, Homby, Ontario, Canada) at 4° C. for 1 hour. The cells were then pelleted by centrifugation, resuspended in rabbit serum (Low-Tox-M rabbit complement; Cedarlane Labs.) as a source of complement, and incubated at 37° C. for 1 hour. Cell suspensions were washed three times and analyzed for efficacy of depletion by flow cytometry using an anti-Thyl.2 Ab, and cell numbers adjusted for injection. Recipient mice received 1000 cGy total body irradiation from a ⁶⁰Co source at a dose rate of 128 cGy/minute on the day of transplant. Bone marrow and spleen cells were given as a single intravenous injection via the tail vein.

Induction of GvHD. GvHD was induced by intravenous injection of a suspension of B6 (H-2^{b}) splenocytes containing 2×10⁶ cells, along with the 2×10⁷ T cell-depleted bone marrow cells described above into irradiated recipients: B10BR (H-2^{k}; principal party) resulting in B6×B10BR mice. B6 mice were also injected with both splenocytes containing 2×10⁶ T cells and 1×10⁷ T cell-depleted bone marrow cells from B6 donors for syngeneic controls.

Photodynamnic treatment. For the purposes of the Kaplan-Meier analysis illustrated in FIGS. 1A and B, B10BR mice were first transplanted with bone marrow and splenocytes from B6 mice. Starting on day 14, some of these mice were sacrificed (B6×B10BR) and their splenocytes (either PDT-treated or not) were administered to other B6×B10BR mice. Splenocytes were obtained from animals that were transplanted in the above conditions. The cells were harvested, washed and resuspended at a density of 1×10⁶ cells/ml in X-VIVO 15™ medium (Bio-Whittaker, Walkersville, Md.) supplemented with 2.5% FBS. The cells were then allowed to internalize 10 μM TH9402 for 40 minutes at 37° C. After a wash with X-VIVO 15 medium supplemented with 10% FBS, the photosensitizer was cleared from cells for 50 minutes. At the end of effiux time, the samples were submitted to photodynamic therapy with 5 J/cm² of light energy at a wavelength 514 nm, and using a sample thickness of 1.7 mm. Four million T cells of the PDT treated or PDT untreated group were injected into recipient mice weekly for 4 weeks starting on day 14 post-transplantation. A second group of animals received 0.4×10⁶ T cells obtained again from spleens of animals with GvHD according to the same infusion schedule. As controls, one group of animals received only culture medium (RPMI-1640), and one group of syngeneic mice (B10BR (H-2k) in B10BR (H-2k)) received cells with or without PDT treatment on the same days as the GvHD groups. Cell administration was performed every week, starting on day 14, for a total of 4 injections. Mice receiving PDT-treated cells had an improved survival over mice receiving cells that were not PDT-treated (FIG. 1A, P=0.02) indicating that PDT eliminated from the cell graft those cells responsible for causing graft versus-host disease. Treatment with PDT did not affect the survival of control mice receiving cells from syngeneic donors.

Mice which were injected with a lower number of T cells (0.4×10⁶ non-treated T cells) displayed a death rate similar to that of mice from the control group (FIG. 1B). However, the inoculation of 0.4×10⁶ PDT-treated T-cells increased the overall survival of the mice from this group compared to the mice from the control group (p=0.04) and from the non-treated group (p=0.01). Mice which received an autologous transplant (C57BL/6 (H-2^{b})→ C57BL/6 (H-2^{b})) and subsequently received repeated injections of non-treated or PDT-treated T-cells did not demonstrate any sign of toxicity and exhibited 100% survival.

### EXAMPLE 2

### Tumor Vaccination in Mice

The strain of mice B6SJL was used for the evaluation of PDT to induce immuno-protection. In generation of tumor cell lysates, EL-4 cells (American Type Culture Collection, ATCC Accession # TIB-39) were seeded in flasks at 10⁶ cells/ml and exposed to 10 μM TH9402 in serum free DMEM without phenol red medium for 40 minutes, followed by exposure to drug-free medium for 90 minutes, then illuminated with a dose of 10J/cm². Treated cells were incubated overnight. After incubation, cells and supernatants were collected and spun down. The resulting supernatant was collected, concentrated by vacuum speed using a molecular sieve (centriplus 3000 molecular weight cut-off), and stored frozen at −70° C. until use.

Six to eight-week old mice were vaccinated subcutaneously on the shoulder with 40 μl of either lysates or medium only once a week for 4 weeks. The animals were rested for a week and then inoculated on the flanke with 1-3×10⁴ tumor cells. A medium alone (DMEM) group served as untreated control. Once the tumor cells were injected, tumor growth was monitored for 90 days. Animals immunized with the supernatant from PDT-treated cells had a delay in tumor cell appearance, in comparison to animals immunized with medium only (DMEM).The results are presented in FIG. 2. The data indicate that the supernatant from PDT treated cells delayed the appearance of tumor compared to the medium control group. These results are in agreement with Korbelik et al (1996) in which they reported that PDT cell lysates following Photofrin treatment do induce a delayed tumor growth.

EXAMPLE 3

### Tumor Vaccination Using Dendritic Cells Exposed to PDT-Treated Cells

Another strain of mice DBA/2J was used for the evaluation of PDT to induce immuno-protection. In the present article, dendritic cells (DCs) were not PDT-treated and were rather used to present the antigens from tumor cells that were treated with PDT in order to enhance their immunogenic effect. li a first step, DCs were generated using conventional protocols by culturing bone marrow cells from DBA/2J mice in RPMI-1640 medium supplemented with GM-CSF (10 ng/ml) and Interleukin-4 (20 ng/ml) for 6 days. DCs were isolated by placing cultured cells over 14.5% metrizamide, and performing differential centrifugation (2400 rpm for 20 minutes). Isolated DCs were then placed in contact with the P815 mastocytoma tumor cell line that had undergone PDT. For PDT, the P815 cells (American Type Culture Collection, ATCC Accession # TIB-64) were seeded in flasks at 10⁶ cells/ml and exposed to 5 μM TH9402 in serum free DMEM without phenol red medium for 40 minutes, followed by exposure to drug-free medium for 50 minutes, then illuminated with a dose of 5 J/cm². PDT-treated P815 cells (3 million cells) were incubated overnight in presence of dendritic cells (1 million cells) in medium used for the production of DCs. After approximately 18 hours of incubation, cells and supernatants Were collected and spun down.

Six to eight-week old mice were vaccinated subcutaneously on the shoulder once a week for 3 weeks with the cell mixture comprised of DCs exposed to PDT-treated P815 cells (total of 2.5-3.0×10⁵ cells). A group of animals were immunized at the same timepoints with dendritic cells alone (DCs generated under the same conditions but not exposed to PDT-treated P815 cells) and served as untreated control. The animals were rested for a week and then inoculated on the flanlk with 1-3×10⁴ tumor cells. Once the tumor cells were injected, tumor growth was monitored for 90 days. Animals immunized with the DCs exposed to PDT-treated cells remained tumor-free for the whole observation period. In contrast, most of the animals (80%) demonstrated tumor recurrence within the same observation period. The results are presented in FIG. 3. The data indicate that whole PDT-treated tumor cells promote a vaccination effect when used in conjunction with dendritic cells. The same strategy could also be amplified using growth factors, such as GM-CSF, or other immunostimulatory molecules, such as interferon and interleukin-2, to promote the immunomodulatory effect of PDT.

While the invention has been described with particular reference to the illustrated embodiment, it will be understood that numerous modifications thereto will appear to those skilled in the art. Accordingly, the above description and accompanying drawings should be taken as illustrative of the invention and not in a limiting sense.

1-48. (canceled)

49. An immunologic vaccine comprising PDT-treated cells or fragments thereof or a supernatant thereof, wherein said cells are treated with a photoactivatable compound selected from the group consisting of: 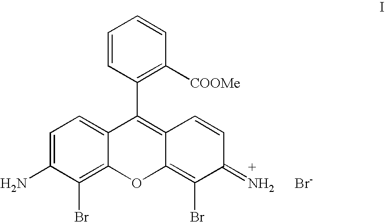
4,5-dibromorhodamine 123 hydrobromide (2′-(6-amino-4,5-dibromo-3-imino-3H-xanthen-9-yl)-benzoic acid methyl ester hydrobromide), 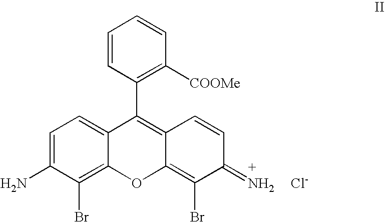
4,5-dibromorhodamine 123 hydrochloride (2′-(6-amino-4,5-dibromo-3-imino-3H-xanthen-9-yl)-benzoic acid methyl ester hydrochloride), 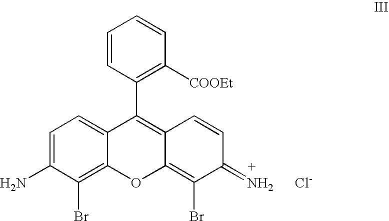
4,5-dibromorhodamine 110 ethyl ester hydrochloride (2′-(6-amino-4,5-dibromo-3-imino-3H-xanthen-9-yl)benzoic acid ethyl ester hydrochloride), 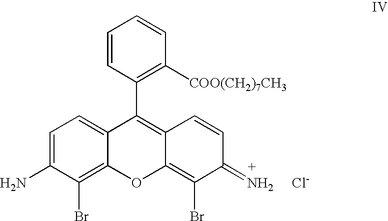
4,5-dibromorhodamine 110 octyl ester hydrochloride (2′-(6-amino-4,5-dibromo-3-imino-3H-xanthen-9-yl)benzoic acid octyl ester hydrochloride), 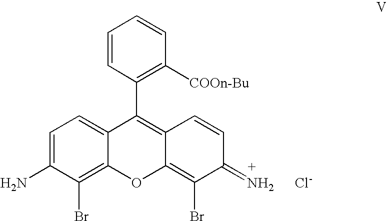
4,5-dibromorhodamine 110 n-butyl ester hydrochloride (2′-(6-amino-4,5-dibromo-3-imino-3H-xanthen-9-yl)benzoic acid n-butyl ester hydrochloride), 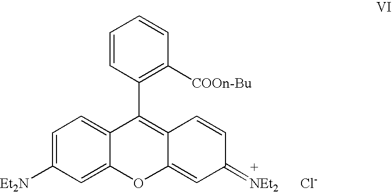
rhodamine B n-butyl ester hydrochloride (2′-(6-diethyl amino-3-diethyl imino-3H-xanthen-9-yl)benzoic acid n-butyl ester hydrochloride), 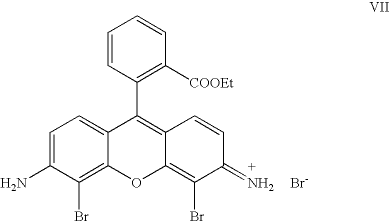
4,5-dibromorhodamine 110 ethyl ester hydrobromide (2′-(6-amino-4,5-dibromo-3-imino-3H-xanthen-9-yl)benzoic acid ethyl ester hydrobromide), 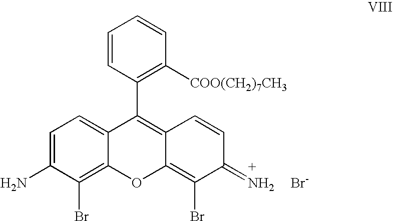
4,5-dibromorhodamine 110 octyl ester hydrobromide (2′-(6-amino-4,5-dibromo-3-imino-3H-xanthen-9-yl)benzoic acid octyl ester hydrobromide), 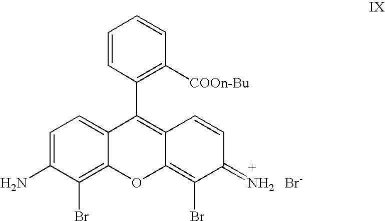
4,5-dibromorhodamine 110 n-butyl ester hydrobromide (2′-(6-amino-4,5-dibromo-3-imino-3H-xanthen-9-yl)benzoic acid n-butyl ester hydrobromide), 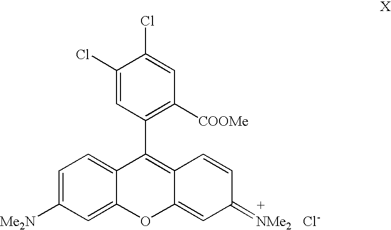
4′,5′-dichlorotetramethylrhodamine (2′-(6-dimethylamino-3-dimethylimino-3H-xanthen-9-yl)-4′,5′-dichloro benzoic acid methyl ester hydrochloride), 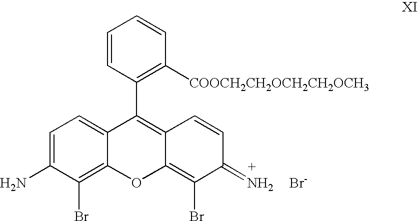
4,5-dibromorhodamine 110 2-(2-methoxy ethoxy)ethyl ester hydrobromide (2′-(6-amino-4,5-dibromo-3-imino-3H-xanthen-9-yl)-benzoic acid 2-(2-methoxy ethoxy) ethyl ester hydrobromide), 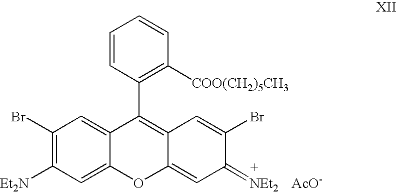
2,7-dibromorhodamine B hexyl ester acetate (2′-(2,7-dibromo-6-diethyl amino-3-diethyl imino-3H-xanthen-9-yl)benzoic acid hexyl ester acetate), 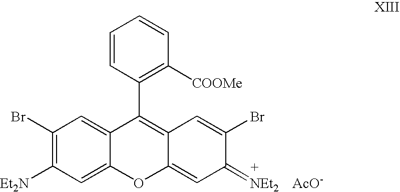
2,7-dibromorhodamine B methyl ester acetate (2′-(2,7-dibromo-6-diethyl amino-3-diethyl imino-3H-xanthen 9-yl)benzoic acid methyl ester acetate), 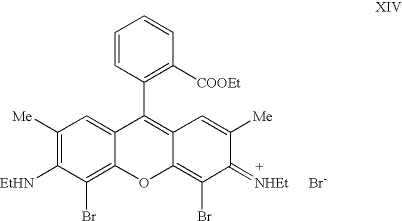
4,5-dibromorhodamine 6G hydrobromide (2′-(4,5-dibromo-2,7-dimethyl-6-ethylamino-3-ethylimino-3H-xanthen-9-yl)benzoic acid ethyl ester hydrobromide), 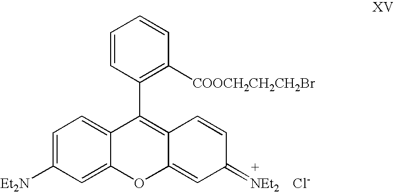
rhodamine B 3-bromopropylester hydrochloride (2′-(6-diethyl amino-3-diethyl imino-3H-xanthen-9-yl)benzoic acid 3-bromopropyl ester hydrochloride), 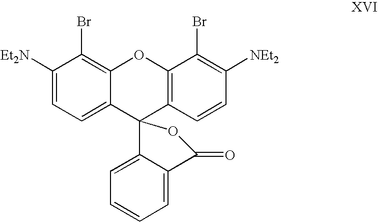
4,5-dibromorhodamine B base (3,3-(4′,5′-dibromo-3′-diethyl amino-6′-diethyl aminoxanthen-9′-yl)-3H-isobenzofuran-1-one), 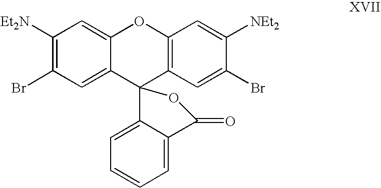
2,7-dibromorhodamine B base (3,3-(2′,7′-dibromo-3′-diethyl amino-6′-diethyl aminoxanthen-9′-yl)-3H-isobenzofuran-1-one), and 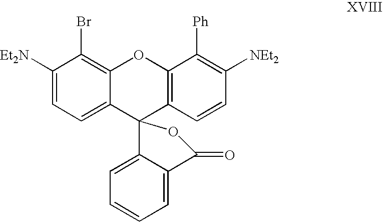
4-bromo-7-phenyl-rhodamine B base (3,3-(4′-bromo-3′-diethyl amino-6′-diethyl amino-5′-phenyl xanthen-9′-yl)-3H-isobenzofuran-1-one) and a pharmaceutically acceptable carrier.

50. The vaccine of claim 49, wherein said photoactivatable compound is selected from the group consisting of 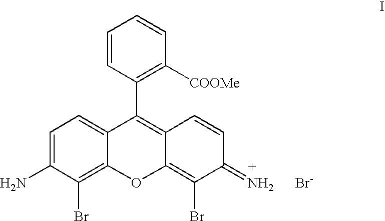
4,5-dibromorhodamine 123 hydrobromide (2′-(6-amino-4,5-dibromo-3-imino-3H-xanthen-9-yl)-benzoic acid methyl ester hydrobromide) and 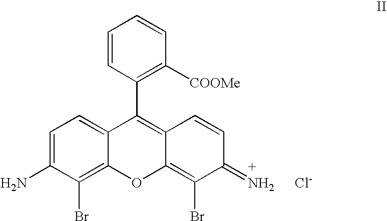
4,5-dibromorhodamine 123 hydrochloride (2′-(6-amino-4,5-dibromo-3-imino-3H-xanthen-9-yl)-benzoic acid methyl ester hydrochloride).

51. The vaccine of claim 49, wherein said compound is activatable by a light having a wavelength in the range of about 400 to about 800 nm.

52. The vaccine of claim 51, wherein said wavelength is in the range of about 450 to about 600 nm.

53. A method of preparing an immunologic pharmaceutical formulation for inhibition, protection or treatment of an immunological disorder, infection or a cancer in an individual, comprising the steps of:
a) treating cells with a photoactivatable compound selected from the group consisting of: 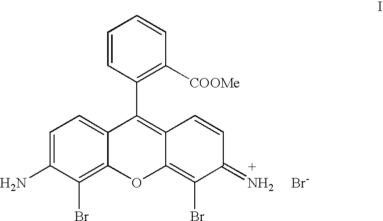
4,5-dibromorhodamine 123 hydrobromide (2′-(6-amino-4,5-dibromo-3-imino-3H-xanthen-9-yl)-benzoic acid methyl ester hydrobromide), 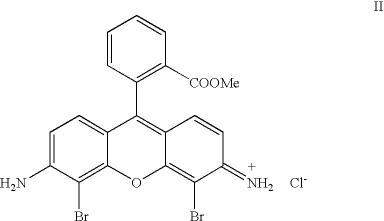
4,5-dibromorhodamine 123 hydrochloride (2′-(6-amino-4,5-dibromo-3-imino-3H-xanthen-9-yl)-benzoic acid methyl ester hydrochloride), 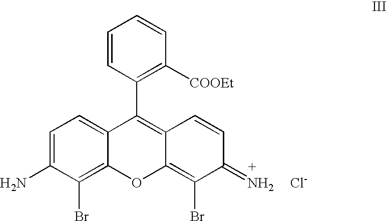
4,5-dibromorhodamine 110 ethyl ester hydrochloride (2′-(6-amino-4,5-dibromo-3-imino-3H-xanthen-9-yl)benzoic acid ethyl ester hydrochloride), 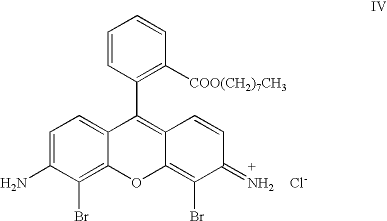
4,5-dibromorhodamine 110 octyl ester hydrochloride (2′-(6-amino-4,5-dibromo-3-imino-3H-xanthen-9-yl)benzoic acid octyl ester hydrochloride), 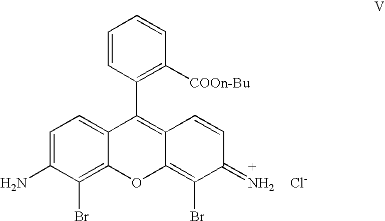
4,5-dibromorhodamine 110 n-butyl ester hydrochloride (2′-(6-amino-4,5-dibromo-3-imino-3H-xanthen-9-yl)benzoic acid n-butyl ester hydrochloride), 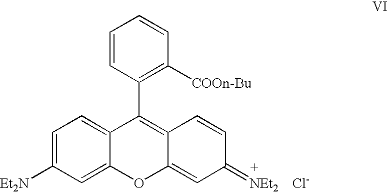
rhodamine B n-butyl ester hydrochloride (2′-(6-diethyl amino-3-diethyl imino-3H-xanthen-9-yl)benzoic acid n-butyl ester hydrochloride), 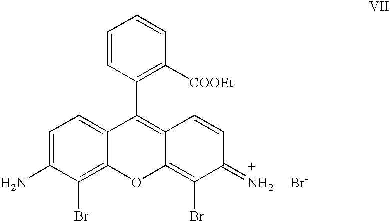
4,5-dibromorhodamine 110 ethyl ester hydrobromide (2′-(6-amino-4,5-dibromo-3-imino-3H-xanthen-9-yl)benzoic acid ethyl ester hydrobromide), 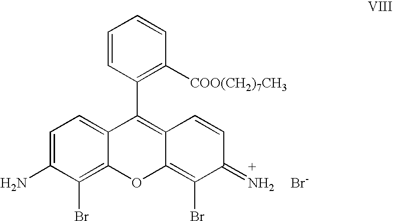
4,5-dibromorhodamine 110 octyl ester hydrobromide (2′-(6-amino-4,5-dibromo-3-imino-3H-xanthen-9-yl)benzoic acid octyl ester hydrobromide), 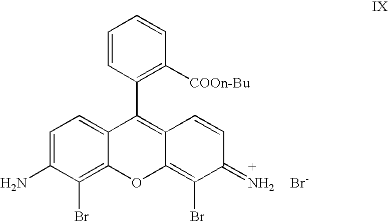
4,5-dibromorhodamine 110 n-butyl ester hydrobromide (2′-(6-amino-4,5-dibromo-3-imino-3H-xanthen-9-yl)benzoic acid n-butyl ester hydrobromide), 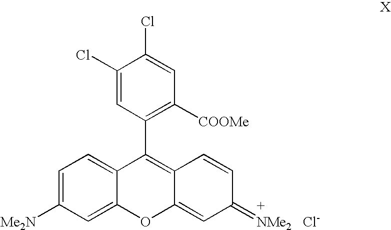
4′,5′-dichlorotetramethylrhodamine (2′-(6-dimethylamino-3-dimethylimino-3H-xanthen-9-yl)-4′,5′-dichloro benzoic acid methyl ester hydrochloride), 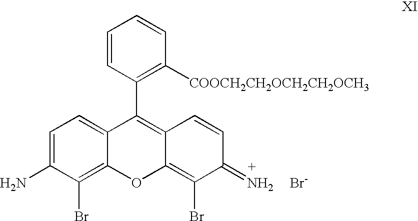
4,5-dibromorhodamine 110 2-(2-methoxy ethoxy)ethyl ester hydrobromide (2′-(6-amino-4,5-dibromo-3-imino-3H-xanthen-9-yl)-benzoic acid 2-(2-methoxy ethoxy) ethyl ester hydrobromide), 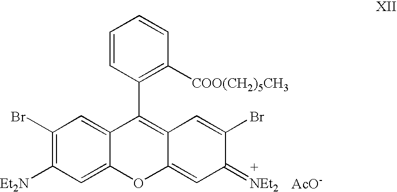
2,7-dibromorhodamine B hexyl ester acetate (2′-(2,7-dibromo-6-diethyl amino-3-diethyl imino-3H-xanthen-9-yl)benzoic acid hexyl ester acetate), 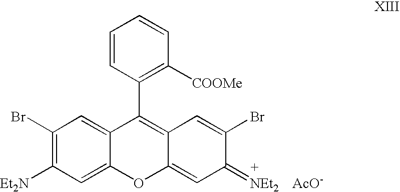
2,7-dibromorhodamine B methyl ester acetate (2′-(2,7-dibromo-6-diethyl amino-3-diethyl imino-3H-xanthen 9-yl)benzoic acid methyl ester acetate), 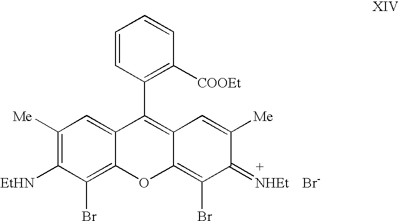
4,5-dibromorhodamine 6G hydrobromide (2′-(4,5-dibromo-2,7-dimethyl-6-ethylamino-3-ethylimino-3H-xanthen-9-yl)benzoic acid ethyl ester hydrobromide), 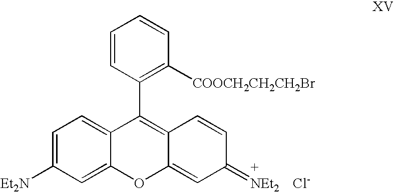
rhodamine B 3-bromopropylester hydrochloride (2′-(6-diethyl amino-3-diethyl imino-3H-xanthen-9-yl)benzoic acid 3-bromopropyl ester hydrochloride), 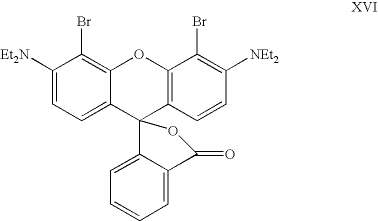
4,5-dibromorhodamine B base (3,3-(4′,5′-dibromo-3′-diethyl amino-6′-diethyl aminoxanthen-9′-yl)-3H-isobenzofuran-1-one), 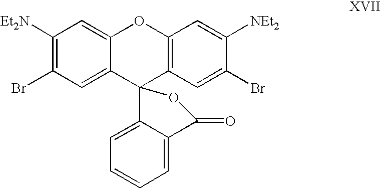
2,7-dibromorhodamine B base (3,3-(2′,7′-dibromo-3′-diethyl amino-6′-diethyl aminoxanthen-9′-yl)-3H-isobenzofuran-1-one), and 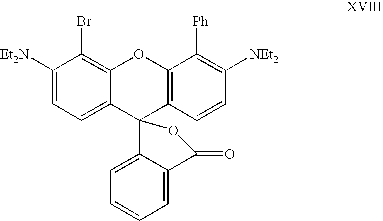
4-bromo-7-phenyl-rhodamine B base (3,3-(4′-bromo-3′-diethyl amino-6′-diethyl amino-5′-phenyl xanthen-9′-yl)-3H-isobenzofuran-1-one). and
b) subjecting said cells to a light to activate said photoactivatable compound, thereby obtaining PDT-treated cells or fragments thereof or a supernatant thereof

54. The method of claim 53, wherein said immunologic pharmaceutical formulation is an autoimmune vaccine.

55. The method of claim 53, wherein said immunological disorder is an alloimmune disorder or an autoimmune disorder.

56. The method of claim 55, wherein said alloimmune disorder is Graft-versus-Host Disease or an organ rejection.

57. The method of claim 55, wherein said autoimmune disease is selected from the group consisting of Rheumatoid Arthritis, Multiple Sclerosis, Scleroderma, Lupus, Autoimmune Hemolytic Anemia, Diabetes Mellitus, Progressive Systemic Sclerosis, Idiopathic Thrombocytopenic Purpura, Psoriasis, Ulcerative Colitis and Crohn's Disease.

58. The method of claim 53, wherein said infection is caused by a bacteria, a virus, a parasite, a fungus, a prion or a protozoan.

59. The method of claim 58, wherein said virus is selected from the group consisting of Human Immunodeficiency Virus (HIV), Hepatitis C Virus (HCV), Hepatitis B Virus (HBV), Human Herpes Virus Type I or II, and Varicella Zoster.

60. The method of claim 53, wherein said infection causes Chagas' Disease.

61. The method of claim 53, wherein said cancer is selected from the group consisting of solid tumors and hematologic tumors.

62. The method of claim 61, wherein said solid tumors are of breast cancer, lung cancer, gastrointestinal cancer, skin cancer or of genitourinary, neurological, head and neck or musculoskeletal origin.

63. The method of claim 61, wherein said hematologic tumors are lymphomas, leukemias, myelomas, myelodysplasias or plasma cell dyscrasias.

64. The method of claim 53, wherein said treatment of said individual cells is effected ex vivo.

65. The method of claim 64, wherein said treatment is an ex vivo treatment effected by perfusion.

66. The method of claim 53, wherein said treatment of said individual cells is effected in vivo.

67. The method of claim 53, wherein said photoactivatable compound is selected from the group consisting of: 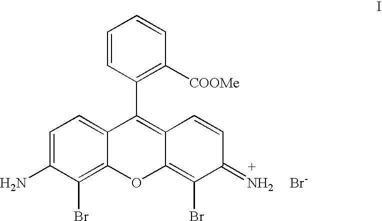
4,5-dibromorhodamine 123 hydrobromide (2′-(6-amino-4,5-dibromo-3-imino-3H-xanthen-9-yl)-benzoic acid methyl ester hydrobromide) and 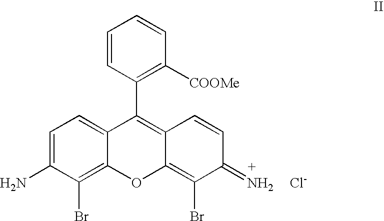
4,5-dibromorhodamine 123 hydrochloride (2′-(6-amino-4,5-dibromo-3-imino-3H-xanthen-9-yl)-be nzoic acid methyl ester hydrochloride).

68. The method of claim 53, wherein said light has a wavelength in the range of about 400 to about 800 nm.

69. The method of claim 68, wherein said wavelength is in the range of about 450 to about 600 nm.

70. A method of inhibiting or treating an immunological disorder, infection or a cancer in an individual, the method comprising the step of administering to an individual in need thereof an effective amount of a pharmaceutical formulation according to claim 53.

71. The method of claim 70, wherein said immunologic disorder is an autoimmune disorder.

72. The method of claim 70, wherein said immunological disorder is an alloimmune disorder.

73. The method of claim 72, wherein said alloimmune disorder is Graft-versus-Host Disease or an organ rejection.

74. The method of claim 71, wherein said autoimmune disorder is selected from the group consisting of Rheumatoid Arthritis, Multiple Sclerosis, Scleroderma, Lupus, Autoimmune Hemolytic Anemia, Diabetes Mellitus, Progressive Systemic Sclerosis, Idiopathic Thrombocytopenic Purpura, Psoriasis, Ulcerative Colitis and Crohn's Disease.

75. The method of claim 70, wherein said infection is caused by a bacteria, a virus, a parasite, a fungus, a prion or a protozoan.

76. The method of claim 75, wherein said virus is selected from the group consisting of Human Immunodeficiency Virus (HIV), Hepatitis C Virus (HCV), Hepatitis B Virus (HBV), Human Herpes Virus Type I or II, and Varicella Zoster.

77. The method of claim 70, wherein said infection causes Chagas' Disease.

78. The method of claim 70, wherein said cancer is selected from the group consisting of solid tumors and hematologic tumors.

79. The method of claim 78, wherein said solid tumors are of breast cancer, lung cancer, gastrointestinal cancer, skin cancer or of genitourinary, neurological, head and neck or musculoskeletal origin.

80. The method of claim 78, wherein said hematologic tumors are lymphomas, leukemias, myelomas, myelodysplasias or plasma cell dyscrasias.

81. The method of claim 70, wherein said photoactivatable compound is selected from the group consisting of: 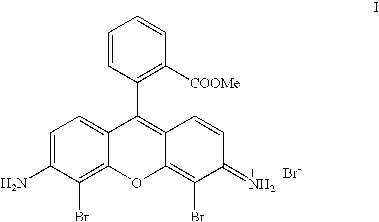
4,5-dibromorhodamine 123 hydrobromide (2′-(6-amino-4,5-dibromo-3-imino-3H-xanthen-9-yl)-benzoic acid methyl ester hydrobromide) and 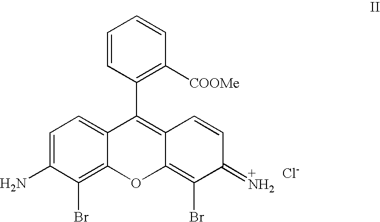
4,5-dibromorhodamine 123 hydrochloride (2′-(6-amino-4,5-dibromo-3-imino-3H-xanthen-9-yl)-benzoic acid methyl ester hydrochloride).
